(19) United States
(12) Patent Application Publication
(10) Pub. No.: US 2022/0223225 A1
(43) Pub. Date: Jul. 14, 2022

(54) CHEMICAL-DISEASE PERTURBATION RANKING

(71) Applicant: Northeastern University, Boston, MA (US)

(72) Inventors: Italo Faria do Valle, Boston, MA (US); Albert-László Barábasi, Brookline, MA (US); Peter Ruppert, Chestnut Hill, MA (US)

(21) Appl. No.: 17/595,185

(22) PCT Filed: May 22, 2020
(86) PCT No.: PCT/US2020/034299

§ 371 Date: Nov. 10, 2021

Related U.S. Application Data
(60) Provisional application No. 62/852,800, filed on May 24, 2019.

(51) Int. Cl. G16B 5/00; G16B 25/10; G16B 40/10; G16H 70/60

(52) U.S. Cl. CPC G16B 5/00; G16H 70/60; G16B 40/10; G16B 25/10

(57) ABSTRACT

Systems and methods of identifying a disease associated with a therapeutic chemical are presented. A method includes generating a candidate disease list based on proximities of proteins associated with a plurality of diseases and proteins associated with a therapeutic chemical in a protein-protein interaction network. The method further includes applying gene expression information associated with the therapeutic chemical to generate enrichment scores for diseases of the candidate disease list and identifying at least one disease associated with the therapeutic chemical based on the determined enrichment scores.

### RELATED APPLICATION

This application claims the benefit of U.S. Provisional Application No. 62/852,800, filed on May 24, 2019. The entire teachings of the above application are incorporated herein by reference.

### GOVERNMENT SUPPORT

This invention was made with government support under 1P01HL132825 from the National Institutes of Health. The government has certain rights in the invention.

### BACKGROUND

Diet can be a key environmental factor that affects human health—while poor diet can significantly increase the risk for coronary heart disease (CHD) and diabetes, a healthy diet can play a protective role, even mitigating genetic risk of CHD. Polyphenols are a class of compounds that can play a protective role for a wide range of diseases, from cancer to diabetes mellitus, as well as for cardiovascular and neurodegenerative diseases. Polyphenols can act as antioxidants and are present in plant-based foods, such as fruits, vegetables, herbs, spices, teas, and wine. Polyphenols are characterized by multiples of phenolic or hydroxy-phenolic structural features, and most contain repeating phenolic moieties of resorcinol, pyrocatechol, pyrogallol, and phloroglucinol linked by ester or carbon-carbon bonds. Recent efforts profiling over 500 polyphenols in more than 400 foods have documented the high diversity of polyphenols humans are exposed to through their diet, ranging from flavonoids to phenolic acids, lignans, and stilbens.

While polyphenols, as one example of a class of chemical compounds that can affect human health, are generally known to provide for healthful effects, underlying molecular mechanisms through which specific polyphenols exert their function, as well as associations with particular diseases, remain largely unexplored.

### SUMMARY

Systems and methods are described that can be used as tools in providing for the identification of diseases affected by a given chemical or class of chemicals, such as polyphenols. The systems and methods described can provide for mechanistic insight as to the molecular pathways responsible for the health implications of a chemical.

A method of identifying a disease associated with a therapeutic chemical includes generating a candidate disease list based on proximities of proteins associated with a plurality of diseases and proteins associated with a therapeutic chemical in a protein-protein interaction network. The method further includes applying gene expression information associated with the therapeutic chemical to generate enrichment scores for diseases of the candidate disease list and identifying at least one disease associated with the therapeutic chemical based on the determined enrichment scores.

A method of filtering data in a protein-protein interaction network includes mapping proteins associated with a plurality of diseases and proteins associated with a therapeutic chemical. The method further includes determining proximities of proteins associated with the plurality of diseases and proteins associated with the therapeutic chemical. An enrichment score is generated for each of the plurality of diseases based on gene expression information associated with the therapeutic chemical. A reduced dataset of proteins within the protein-protein interaction network is generated, the reduced dataset of proteins being proteins associated with a subset of the plurality of diseases based on the determined proximities and the determined enrichment scores. The subset of diseases can be a candidate disease list.

Generating a candidate disease list can include generating a proximity value for a disease and the therapeutic chemical. Determining proximities, or determining a proximity value, can be based on shortest path lengths between nodes representing proteins associated with the disease and nodes representing proteins associated with the therapeutic chemical in the protein-protein interaction network. The proximity value can be a distance metric, such as d_{c}(S,T) as given by the following:

$${d}_{c} ( S , T ) = \frac{1}{‖ T ‖} {\sum }_{t ∈ T} {min}_{s ∈ S} d ( s , t ) \qquad [ 1 ]$$

where S is a set of proteins associated with the disease, T is a set of proteins associated with the therapeutic chemical, s is a node representing a protein in set S, t is a node representing a protein in set T, and d(s,t) is a shortest path length between nodes s and tin the protein network.

Generating an enrichment score can include measuring an extent of gene expression perturbation by the therapeutic chemical for a disease, such as, for example, by performing a Gene Set Enrichment Analysis.

The methods can further include ranking the diseases of the candidate disease list based on the determined proximity and the determined enrichment scores. The protein-protein interaction network can be a human interactome. The proteins associated with a therapeutic chemical can be proteins to which the therapeutic chemical binds. For example, the therapeutic chemical can be a polyphenol and the proteins associated with the therapeutic chemical can be binding targets of the polyphenol.

A method of treating a subject having a disease includes administering a therapeutic chemical, wherein the disease is a disease identified by any of the method described above as being associated with the therapeutic chemical.

A system for identifying a disease associated with a therapeutic chemical includes a processor configured to generate a candidate disease list based on proximities of proteins associated with a plurality of diseases and proteins associated with a therapeutic chemical in a protein-protein interaction network. The processor is further configured to apply gene expression information associated with the therapeutic chemical to generate enrichment scores for diseases of the candidate disease list and to identify at least one disease associated with the therapeutic chemical based on the determined enrichment scores.

A system for filtering data in a protein-protein interaction network includes a processor configured to map proteins associated with a plurality of diseases and proteins associated with a therapeutic chemical and determine proximities of proteins associated with the plurality of diseases and proteins associated with the therapeutic chemical. The processor is further configured to generate an enrichment score for each of the plurality of diseases based on gene expression information associated with the therapeutic chemical and to generate a reduced dataset of proteins within the protein-protein interaction network, the reduced dataset of proteins being proteins associated with a subset of the plurality of diseases based on the determined proximities and the determined enrichment scores.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is diagram of a filter for reducing proteins of a protein-protein interaction network for a therapeutic chemical.

FIG. 2 is a diagram of a computer processor operation 100 for identifying a disease associated with a therapeutic chemical.

FIG. 3 is a schematic view of a computer network environment in which embodiments of the present invention may be deployed.

FIG. 4 is a block diagram of computer nodes or devices in the computer network of FIG. 3.

FIG. 5A is a schematic representation of an interactome, with highlighted regions where polyphenol targets and disease proteins are localized.

FIG. 5B is a diagram showing the selection criteria of the polyphenols evaluated in a study.

FIG. 5C is a distribution of the number of polyphenol protein targets mapped to the human interactome.

FIG. 5D is a graph of the top (n=15) enriched Gene Ontology (GO) pathways (Biological Process) among all polyphenol protein targets. The X axis shows the proportion of targets mapped to each pathway.

FIG. 5E is a plot of the size of the Largest Connected Component (LCC) formed by the targets of each polyphenol in the interactome and the corresponding significance (z-score).

FIG. 6 illustrates protein subgraphs of the targets of twenty-three polyphenols. The targets of the twenty-three polyphenols form connected components in the interactome. For example, piceatannol targets form a unique connected component of 23 proteins, while quercetin targets form multiple connected components, the biggest with 140 proteins. Polyphenol targets that are not connected to any other target are not shown in the figure.

FIG. 7A illustrates an interactome neighborhood showing EGCG protein targets and their interactions with type 2 diabetes (T2D)-associated proteins.

FIG. 7B is a distribution of AUC values considering the predictions of therapeutic effects for 65 polyphenols.

FIG. 7C is illustrates a comparison of the ECGC-disease associations considering the CTD database and the in-house database derived from the manual curation of the literature.

FIG. 7D is a graph of a comparison of the prediction performance when considering known EGCG-disease associations from the CTD, in-house manually curated database, or combined datasets.

FIG. 8A is a schematic representation of the relationship between the extent to which a polyphenol perturbs disease genes expression, its proximity to the disease genes, and its therapeutic effects.

FIG. 8B illustrates an interactome neighborhood showing the modules of Skin Diseases (SK), Genistein, and Cerebrovascular Disorders (CD). The SK module has 10 proteins with high perturbation scores (>2) in the treatment of the MCF7 cell line with 1 μM of genistein. Genes associated to SK are significantly enriched among the most differentially expressed genes, and the maximum perturbation score among disease genes is higher in SK than CD.

FIG. 8C illustrates therapeutic associations for four polyphenols. Among the diseases in which genes are enriched with highly perturbed genes, those with therapeutic associations show smaller network distances to the polyphenol targets than those without. The same trend is observed in treatments of the polyphenols quercetin, resveratrol, and myricetin.

FIG. 9A is a schematic representation of proximal and distal diseases in relation to genistein targets. Each node represents a disease and the node size proportional to the perturbation score after treatment with genistein (1 μM, 6 hours). Distance from the origin represents the network proximity (dc) to genistein targets. Purple nodes represent diseases in which the therapeutic association was previously known.

FIG. 9B illustrates cumulative distributions of the maximum perturbation scores of genes from diseases that are distal or proximal to polyphenol targets considering different polyphenols (1 μM, 6 hours): genistein, quercetin, resveratrol, and myricetin. Statistical significance was evaluated with the Kolmogorov Smirnov test.

FIG. 10 illustrates an interactome neighborhood containing the interactions between proteins associated with Vascular Diseases and the targets of 1,4-naphthoquinone, gallic acid, and rosmarinic acid.

FIG. 11A illustrates an interactome neighborhood showing Rosmarinic acid (RA) targets and the RA-VD-platelet module—the connected component formed by the RA target FYN and the VD proteins associated to platelet function PDE4D, CD36, and APP—and the receptor of platelet stimulants used in our experiments (Collagen/CRPXL, TRAP6, U46619, and ADP).

FIG. 11B is a graph of average shortest path length from each platelet stimulant receptor and the RA-VD-platelet module formed by the proteins FYN, PDE4D, CD36, APP.

FIG. 11C is a graph of assessed aggregation of platelets. Platelet-rich plasma (PRP) or washed platelets were pre-treated with RA for 1 hour before stimulation with either collagen (1 μg/mL), collagen-related peptide (CRP-XL, 1 μg/mL), thrombin receptor activator peptide-6 (TRAP-6, 20 μM), U46619 (1 μM), or ADP (10 μM).

FIG. 11D is a graph of assessed alpha granule secretion of the platelets of FIG. 11C.

FIG. 11E illustrates results of protein tyrosine phosphorylation (P-Tyr) assessment of the platelets of FIG. 11C. Numbers on the right indicate protein molecular weight. N=3-6 separate blood donations, mean +/−SEM.

FIG. 11F illustrates results of protein tyrosine phosphorylation (P-Tyr) assessment of the platelets of FIG. 11C.

### DETAILED DESCRIPTION

A description of example embodiments follows.

Systems and methods are presented for identifying diseases whose proteins are candidates to show gene expression perturbation under a treatment with a given chemical compound. The systems and methods presented herein can function as a filter in a protein-protein interaction network, such as the human interactome, to reduce proteins present in the network to a subset of proteins associated with a chemical compound and a disease.

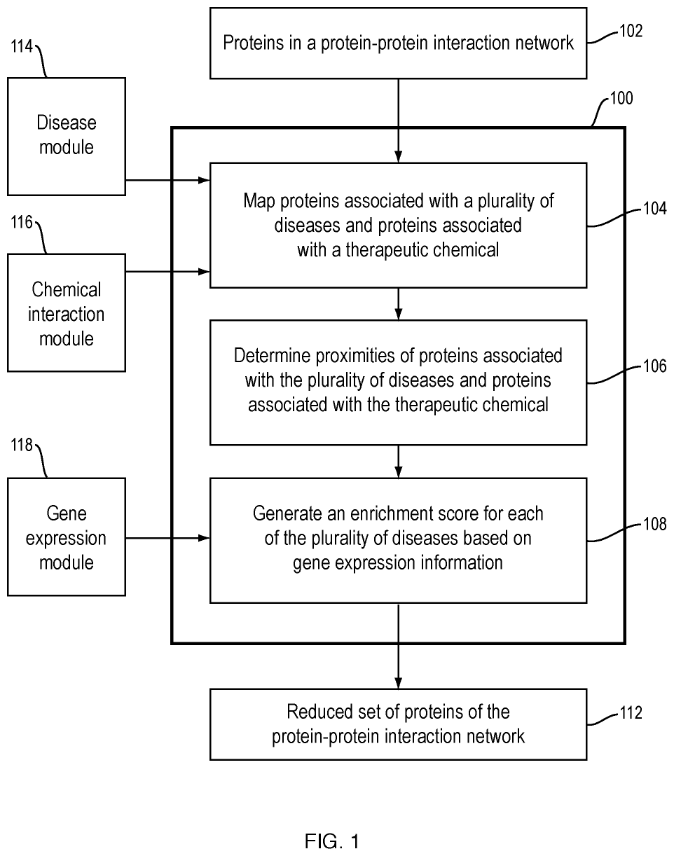

An example of a filter 100 that can be applied to a protein-protein interaction network 102 is shown in FIG. 1. From the proteins present in a protein-protein interaction network 102, the filter 100 functions to reduce the proteins present in the network to a subset of proteins that are associated with a chemical-disease relationship. Systems and methods including filter 100 operate by mapping proteins associated with a plurality of diseases and proteins associated with a therapeutic chemical (step 104). Information regarding proteins associated with one or more diseases can be provided from a disease module 114 to identify disease clusters within the protein-protein interaction network. Information regarding proteins associated with one or more chemicals can be provided by a chemical interaction module 116 to identify chemical target locations within the network. After mapping, the filter 100 determines proximities, within the network, of proteins associated with the plurality of diseases and proteins associated with the therapeutic chemical (step 106). Gene expression information is applied to generate an enrichment score for each of the one or more diseases under consideration (step 108). The gene expression information can be provided by a gene expression module 118 that includes perturbation signatures for cell lines treated with the one or more chemicals. Based on the determined proximities and enrichment scores, the proteins within the network are reduced to one or more sets 112 associated with a particular chemical-disease relationship.

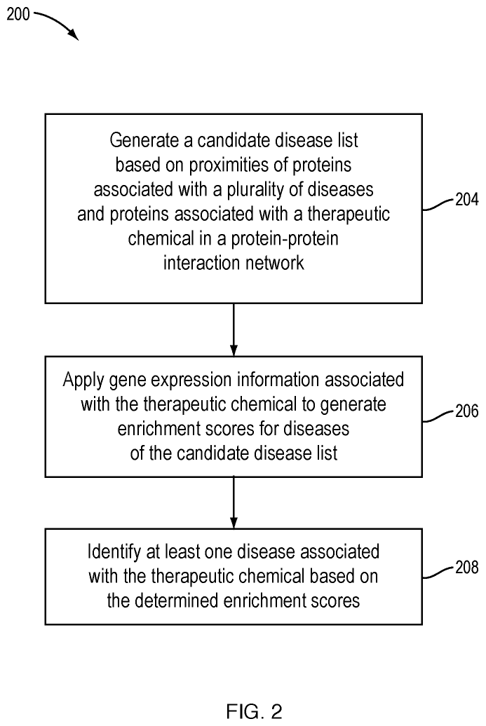

An example of a method 200 for identifying a disease associated with a therapeutic chemical is shown in FIG. 2. The method includes generating a candidate disease list based on proximities of proteins associated with a plurality of diseases and proteins associated with a therapeutic chemical in a protein-protein interaction network (step 204). Gene expression information can be applied to generate an enrichment score for diseases of the candidate disease list (step 206). From the determined enrichment scores of diseases in the candidate disease list, at least one diseases associated with the therapeutic chemical can be identified (step 208).

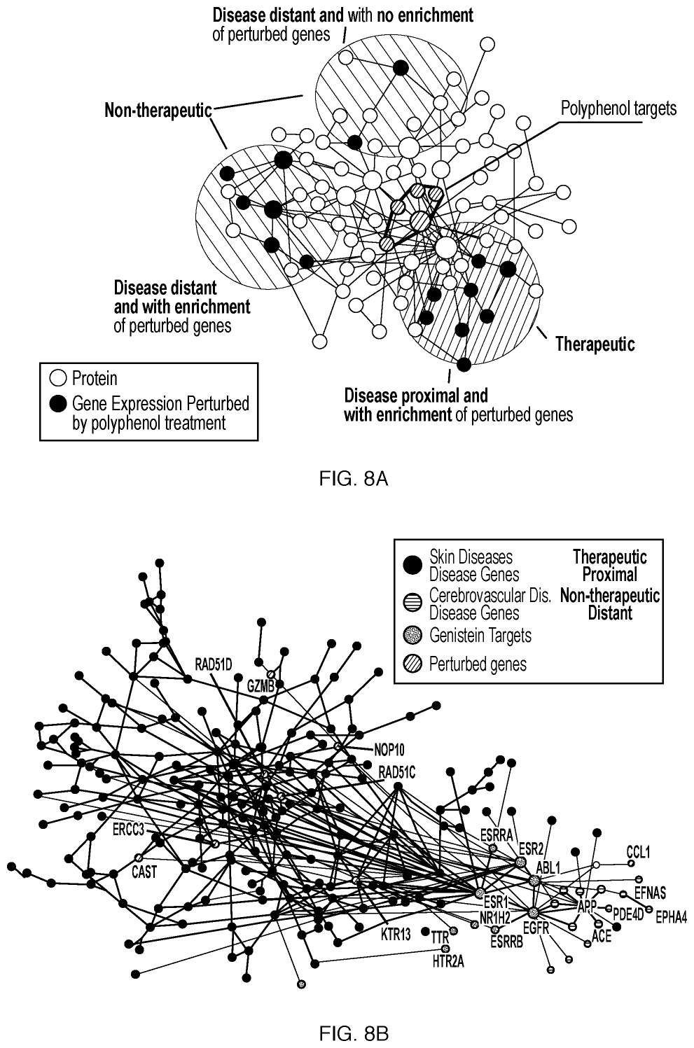

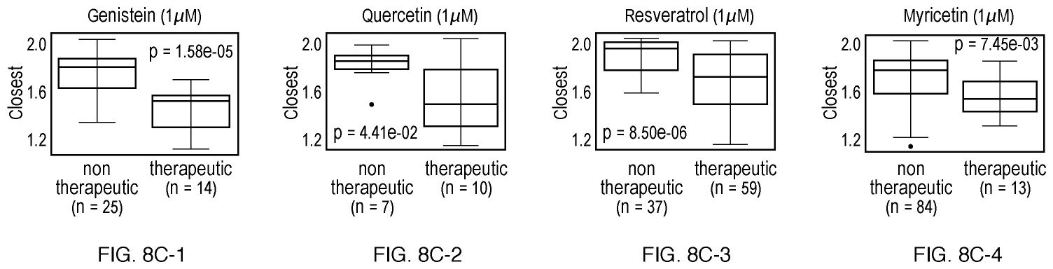

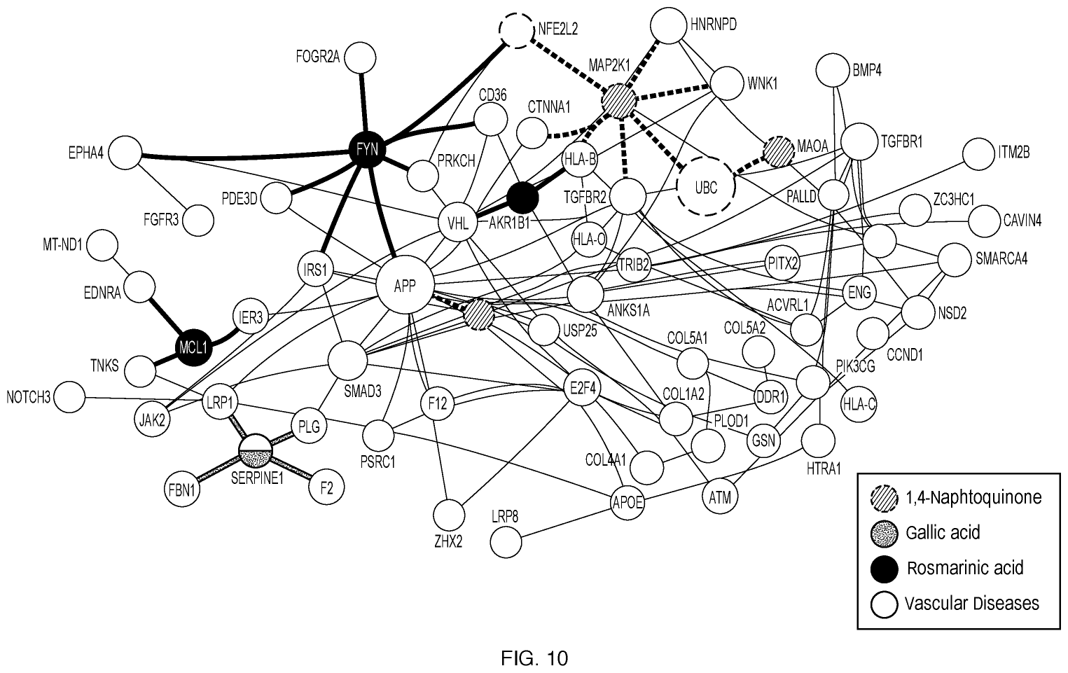

Example methods and systems for identifying a disease cluster within a protein network are described in WO2015/084461, the entire contents of which are incorporated herein by reference. Disease clusters identified within a network can be used to generate candidate disease lists. Examples of disease clusters within a network are described in the examples that follow and are shown, for example, in FIGS. 8A, 8B and 10.

The chemical compound can be any chemical, including, for example natural and food-borne chemical compounds, therapeutic chemicals, such as polyphenols, synthetic drugs, and nutraceuticals, and nontherapeutic chemicals, such as toxins, and general phytochemicals present in food. In the examples that follow, polyphenols are described for illustration purposes only.

The protein-protein interaction network can be, for example, the human interactome, which includes a map of protein interactions in the human cell. Other protein-protein interaction networks can be used, such as, for example, networks from STRINGDB and GeneMania databases.

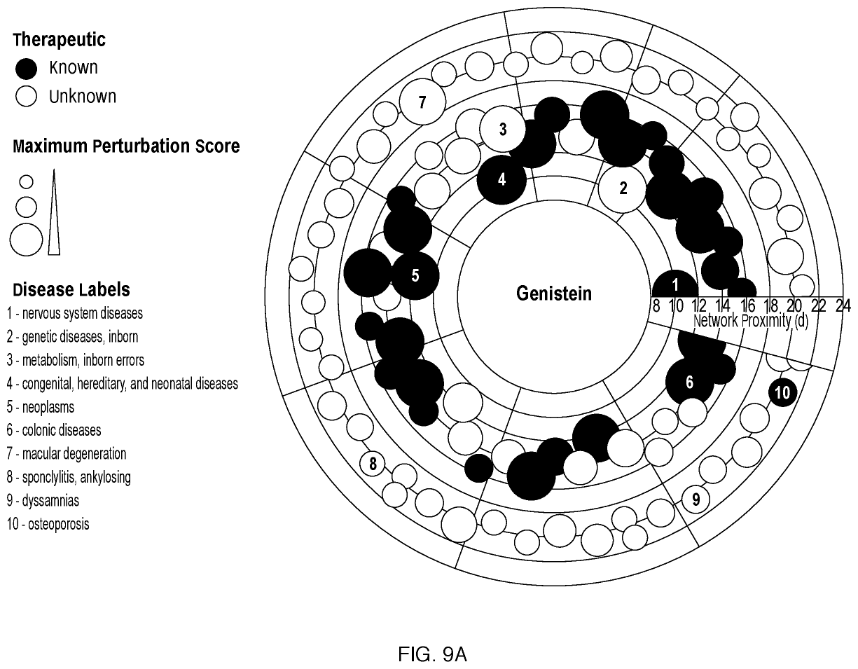

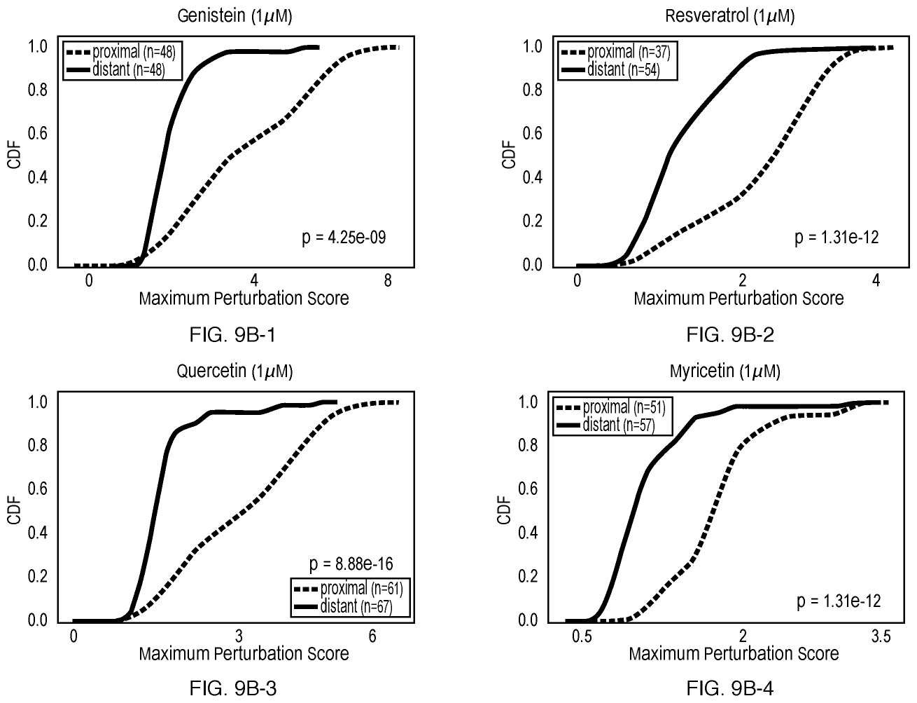

In the systems and methods shown in FIGS. 1 and 2, where several diseases and/or several chemicals are considered, a Chemical-Disease Perturbation Ranking (CDPR) can be produced. The CDPR can provide for identification of chemical compounds that can be used for disease treatment or that present health-related effects, while also providing for mechanistic information of chemical-disease relationships. Examples of disease clusters within a network are described in the examples that follow and are shown, for example, in FIG. 9A.

As further described in the examples that follow, generating the candidate disease list can include generating a proximity value for a disease and the therapeutic chemical. Proximity between a disease and a chemical can be evaluated using a distance metric that takes into account path lengths between chemical targets and disease proteins within the network. For example, the proximity value can be determined based on shortest path lengths between nodes representing proteins associated with the disease and nodes representing proteins associated with the therapeutic chemical. The proximity value can be a distance metric d_{c}(S,T) determined according to:

$${d}_{c} ( S , T ) = \frac{1}{‖ T ‖} {\sum }_{t ∈ T} {min}_{s ∈ S} d ( s , t ) \qquad [ 1 ]$$

where S is a set of proteins associated with the disease, T is a set of proteins associated with the therapeutic chemical, s is a node representing a protein in set S, t is a node representing a protein in set T, and d(s,t) is a shortest path length between nodes s and tin the protein network.

To assess significance of a distance between a chemical and a disease (S,T), a reference distance distribution corresponding to expected distances between two randomly selected groups of proteins matching size and degrees of the original disease proteins and chemical targets in the network can be used. For example, a reference distance distribution can be generated by calculating a proximity between two randomly selected groups, and this procedure can be repeated several (e.g., 100, 500, 1000, 2000) times. The mean and standard deviation of the reference distribution can be used to convert the absolute distance to a relative distance (Z-score). Due to the scale-free nature of the human interactome, there are few nodes with high degrees. To avoid repeatedly choosing the same (high degree) nodes, a degree-preserving random selection can be performed.

As further described in the examples that follow, generating an enrichment score for diseases of a candidate disease list can include measuring an extent of gene expression perturbation by the therapeutic chemical for a given disease. This can include performing a Gene Set Enrichment Analysis. For example, pertubations signatures can be obtained, such as from the ConnectivityMap database (https://clue.io/), for cell lines treated with different chemicals. These signatures reflect the perturbation of the gene expression profile caused by treatment with a chemical under consideration relative to a reference population, which is composed of other treatments in the same experimental plate. For chemicals having more than one experimental instance (e.g., time of exposure, cell line, dose), the one with highest distil\_cc\_q75 value (i.e., 75th quantile of pairwise spearman correlations in landmark genes) can be selected. Gene Set Enrichment Analysis can then be performed to evaluate the enrichment of disease genes among the top deregulated genes in the perturbation profiles. This analysis results in an Enrichment Score (ES) that has small values when genes are randomly distributed among the ordered list of expression values and high values when genes are concentrated at the top or bottom of the list. Methods of performing an Enrichment Analysis are further described in Subramanian, A. et al. “Gene set enrichment analysis: a knowledge-based approach for interpreting genome-wide expression profiles.” Proc. Natl. Acad. Sci. U. S. A. 102, 15545-50 (2005), the entire contents of which is incorporated herein by reference.

An ES significance can be calculated by creating, for example, 1000 random selections of gene sets with the same size as the original gene set and calculating an empirical p-value by considering a proportion of random sets resulting in ES smaller than the original case. The p-value can be adjusted for multiple testing by using the Benjamini-Hochberg method.

With the proximity values and enrichment scores, the diseases of the candidate disease list can be ranked to provide the CDPR. For example, the ranking can prioritize chemicals by therapeutic potential. The chemicals with greatest therapeutic potential can be defined as those that are proximal to disease proteins and significantly perturb expression of disease genes. The CDPR can advantageously provide for prioritization of a set of chemicals in respect to a disease, or a set of diseases in respect to a chemical, for further evaluation. The CDPR can also provide for a quantitative and molecular-based description of a relationship between chemical compound targets and disease processes, which can in-turn provide for mechanism-of-action information for the chemical compounds.

Conventional methods of evaluating chemical-disease relations involve evaluation of structural properties of chemical compounds. The methods and systems described can advantageously omit such analysis by accounting for how a chemical interacts with various proteins and how those proteins interact with each other and with associated disease processes through the protein-protein interaction network. The methods and systems described do not require knowledge of the specific type of interactions (e.g., activation, inhibition) between a chemical and its protein targets.

In the case of polyphenols, or other food-borne chemicals, the systems and methods described can advantageously provide for the identification of health effects related to chemical compounds present in foods. For example, and as described in the Example sections that follow, from a CDPR, Rosemarinic Acid (RA) was shown to have an association with vascular diseases and was predicted to have a direct impact on platelet function. With this information, RA was further evaluated, and experimental evidence demonstrated that RA inhibits platelet aggregation and alpha granule secretion, thereby providing for valuable information of foods that may benefit individuals with poor cardiovascular health.

The systems and methods described can advantageously provide for identification of chemical compounds that can be potentially used for disease treatment, identification of health effects related to chemical compounds, such as those present in foods, and streamlining of research by prioritizing chemicals demonstrated to show bioactivity. This methodology can be coupled with technologies such as CRISPR-CAS9 to genetically change life forms (e.g., plants and their seeds) for greater production of chemical compounds with beneficial health effects.

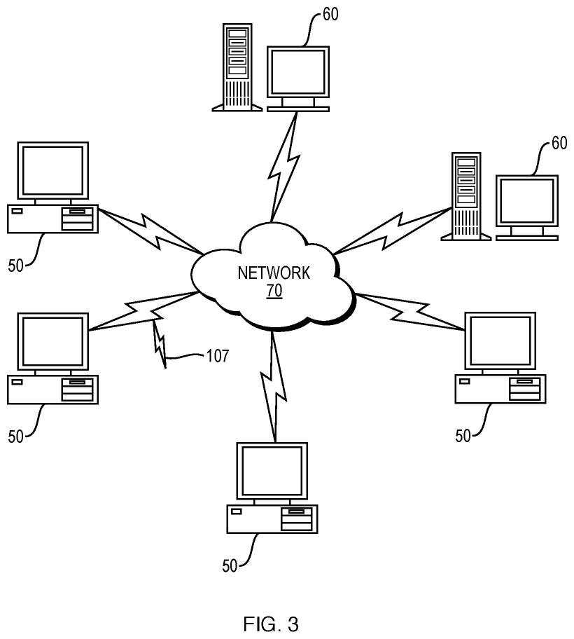

FIG. 3 illustrates a computer network or similar digital processing environment in which the systems and methods described may be implemented. Client computer(s)/devices/exercise apparatuses 50 and server computer(s) 60 provide processing, storage, and input/output devices executing application programs and the like. Client computer(s)/devices 50 can also be linked through communications network 70 to other computing devices, including other client devices/processes 50 and server computer(s) 60. Communications network 70 can be part of a remote access network, a global network (e.g., the Internet), a worldwide collection of computers, cloud computing servers or service, Local area or Wide area networks, and gateways that currently use respective protocols (TCP/IP, Bluetooth, etc.) to communicate with one another. Other electronic device/computer network architectures are suitable.

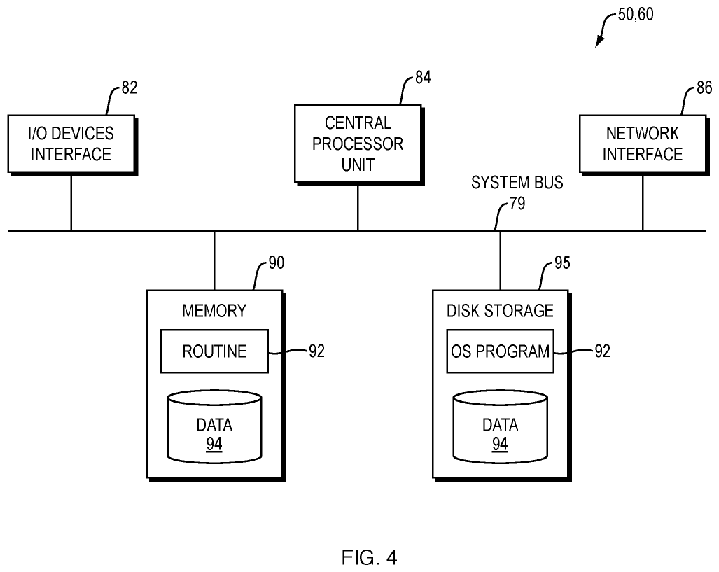

FIG. 4 is a diagram of the internal structure of a computer (e.g., client processor/device 50 or server computers 60) in the computer network of FIG. 3. Each computer 50, 60 contains system bus 79, where a bus is a set of hardware lines used for data transfer among the components of a computer or processing system. Bus 79 is essentially a shared conduit that connects different elements of a computer system (e.g., processor, disk storage, memory, input/output ports, network ports, etc.) that enables the transfer of information between the elements. Attached to system bus 79 is I/O device interface 82 for connecting various input and output devices (e.g., keyboard, mouse, displays, printers, speakers, etc.) to the computer 50, 60. Network interface 86 allows the computer to connect to various other devices attached to a network (e.g., network 70 of FIG. 3). Memory 90 provides volatile storage for computer software instructions 92 and data 94 used to implement embodiments of the present invention (e.g., processor routines and code for creating a directed acyclic graph (DAG) as a function of computed alignment indices and aligning sequence reads against the DAG being developed, as described herein). Disk storage 95 provides nonvolatile storage for computer software instructions 92 and data 94 used to implement an embodiment of the present invention. Central processor unit 84 is also attached to system bus 79 and provides for the execution of computer instructions.

In particular, embodiments of the present invention execute processor routines for the filter 100 and method 200 of FIGS. 1 and 2, respectively. In one embodiment, the processor routines 92 and data 94 are a computer program product (generally referenced 92), including a non-transitory computer readable medium (e.g., a removable storage medium such as one or more DVD-ROM's, CD-ROM's, diskettes, tapes, etc.) that provides at least a portion of the software instructions for the invention system. Computer program product 92 can be installed by any suitable software installation procedure, as is well known in the art. In another embodiment, at least a portion of the software instructions may also be downloaded over a cable, communication and/or wireless connection. In other embodiments, the invention programs are a computer program propagated signal product 107 embodied on a propagated signal on a propagation medium (e.g., a radio wave, an infrared wave, a laser wave, a sound wave, or an electrical wave propagated over a global network such as the Internet, or other network(s)). Such carrier medium or signals provide at least a portion of the software instructions for the present invention routines/program 92.

In alternative embodiments, the propagated signal is an analog carrier wave or digital signal carried on the propagated medium. For example, the propagated signal may be a digitized signal propagated over a global network (e.g., the Internet), a telecommunications network, or other network. In one embodiment, the propagated signal is a signal that is transmitted over the propagation medium over a period of time, such as the instructions for a software application sent in packets over a network over a period of milliseconds, seconds, minutes, or longer. In another embodiment, the computer readable medium of computer program product 92 is a propagation medium that the computer system 50 may receive and read, such as by receiving the propagation medium and identifying a propagated signal embodied in the propagation medium, as described above for computer program propagated signal product.

Generally speaking, the term “carrier medium” or transient carrier encompasses the foregoing transient signals, propagated signals, propagated medium, other mediums and the like.

In other embodiments, the computer program product 92 provides Software as a Service (SaaS) or similar operating platform.

Alternative embodiments can include or employ clusters of computers, parallel processors, or other forms of parallel processing, effectively leading to improved performance, for example, of generating a computational model. Given the foregoing description, one of ordinary skill in the art understands that different portions of processor routine 100 and different iterations operating on respective sequence reads may be executed in parallel on such computer clusters or parallel processors.

### EXEMPLIFICATION

### Example 1: Predicting Health Impact of Dietary Polyphenols Using a Chemical-Disease Perturbation Ranking

Despite the widespread evidence of the positive role of polyphenols on human health, the underlying molecular mechanisms through which specific polyphenols exert their function remain largely unexplored. From a mechanistic perspective their role is rather special because dietary polyphenols are not processed by the endogenous metabolic processes of anabolism and catabolism. Rather, dietary polyphenols impact human health through their ant- or pro-oxidant activity, by binding to proteins and modulating the activity of key cellular signaling and metabolic pathways, interacting with digestive enzymes, and modulating gut microbiota growth. Yet, the variety of experimental settings used so far to explore the molecular effects of polyphenols—represented by different concentrations, administration routes, model organisms, populations, and evaluated outcomes—have, to date, offered a range of often conflicting evidence for interpretation. For example, different clinical trials resulted in contrasting conclusions about the beneficial effects of resveratrol on glycemic control of type 2 diabetes patients. Therefore, there is a need for a framework to interpret the evidence present in the literature, and to offer in-depth mechanistic predictions on the molecular pathways responsible for the health implications of polyphenols present in diet. These insights can aid in the development of novel diagnostic and therapeutic strategies, and may lead to the synthesis of novel drugs.

A network medicine framework was developed to capture the molecular interactions between polyphenols and their cellular binding targets, unveiling their relationship to complex diseases. The developed framework is based on the human interactome, a comprehensive network of all known physical interactions between human proteins, which has been validated before as a platform for understanding disease mechanisms, rational drug target identification, and drug repurposing.

First, it was found that the proteins to which polyphenols bind form identifiable neighborhoods in the human interactome. It was then demonstrated that the proximity between polyphenol targets and proteins associated with specific diseases is predictive of the known therapeutic effects of polyphenols. Finally, the potential therapeutic effects of rosmarinic acid on vascular diseases was unveiled with a prediction that the effect was related to modulation of platelet function. This prediction was confirmed by the performance of experiments that demonstrated that rosmarinic acid modulates platelet function in vitro by inhibiting tyrosine protein phosphorylation. Altogether, the results demonstrate that the network-based relationship between disease proteins and polyphenol targets offers a tool to systematically unveil the health effects of polyphenols.

The methodology described can provide for the foundation of mechanistic interpretation of alternative pathways through which polyphenols can affect health: e.g., the combined effect of different polyphenols and their interaction with drugs. Furthermore, the methodology described can be applied to other food-related chemicals, providing a framework to understand their health effects.

### Example 2: Results: Polyphenol Targets Cluster in Specific Functional Neighborhoods of the Interactome

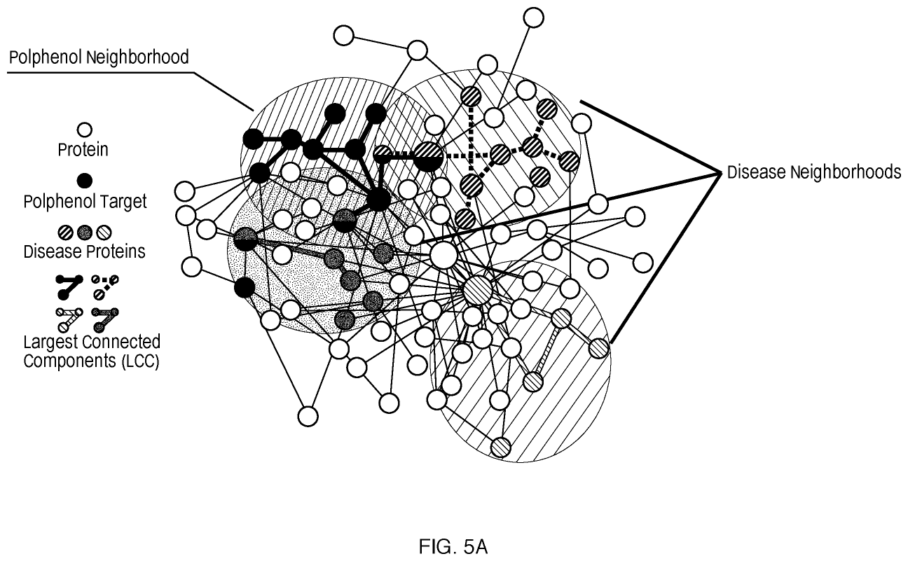

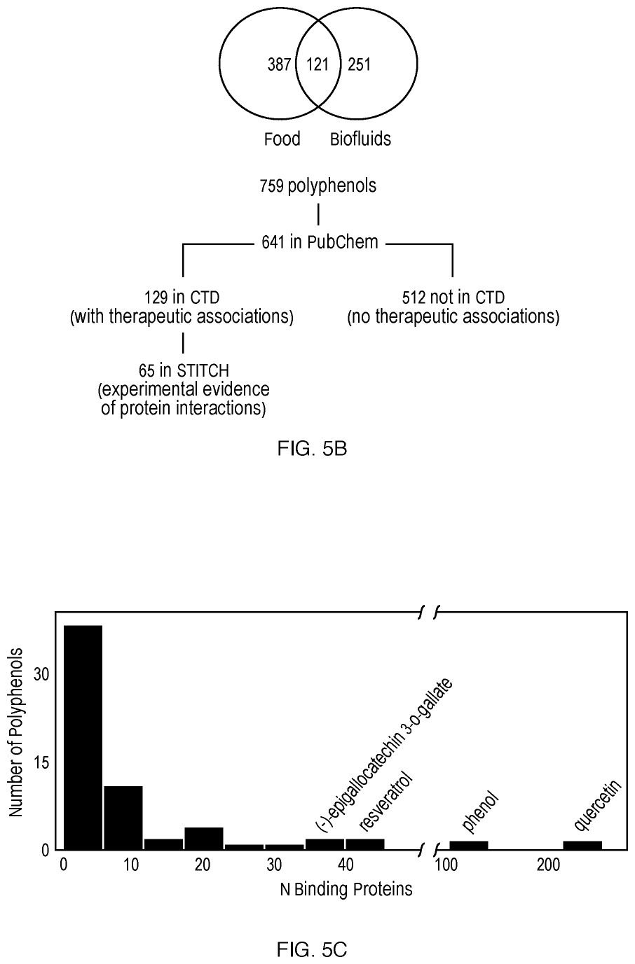

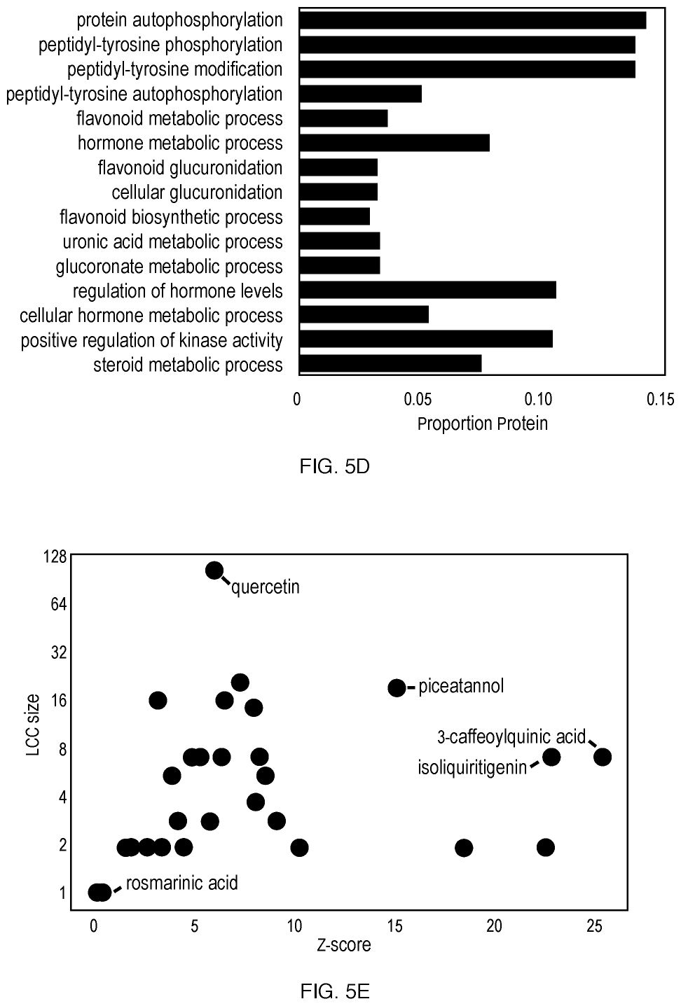

The study started with a list of 759 polyphenols catalogued in the PhenolExplorer database, of which 387 were only detected in foods, 251 were only detected in biofluids, and 121 are present in both foods and biofluids (FIG. 5B). From the list, 118 (15%) polyphenols were removed for which PubChem IDs could not be identified and 512 (67%) that lacked a manually curated ‘therapeutic’ label in the Comparative Toxicogenomics Database (CTD). Of the remaining 129 polyphenols, 65 have experimentally validated protein targets in the STITCH database, providing for the group of polyphenols that were the center of the study. This group represented well-studied polyphenols, from EGCG, the active ingredient of green tea with demonstrated glucose lowering properties, to polyphenols that have the largest number disease associations in CTD. Of these 14 were detected in blood according to the Human Metabolome Database, with maximum concentrations ranging from 10 nM to 80 μM, and, of the remaining 51, 35 were predicted to have high gastrointestinal absorption.

To identify the cellular processes potentially affected by specific polyphenol molecules, the polyphenol targets were mapped to the human interactome, consisting of 17,651 proteins and 351,393 interactions (FIG. 5A). It was found that 19 of the 65 studied polyphenols have only one protein target, while a few polyphenols have an exceptional number of recorded targets, like quercetin (216 targets), phenol (98), resveratrol (63), (−)-epigallocatechin 3-o-gallate (51), and ellagic acid (42) (FIG. 5C). The Jaccard Index (JI) of the protein targets of each polyphenol pair was computed, and only a limited similarity of targets among different polyphenols (average JI=0.0206) was found. Even though the average JI was small, it was still significantly higher (Z=147) than the JI expected if the polyphenol targets were randomly assigned from the pool of all network proteins with degrees matching the original set. This finding suggests that while each polyphenol targets a specific set of proteins, their targets are confined to a common pool of proteins, likely determined by commonalities in the binding domains the three-dimensional structure of the protein targets. Gene Ontology (GO) Enrichment Analysis of all polyphenol protein targets revealed that they tend to target pathways related to post-translation protein modifications, regulation, and xenobiotic metabolism (FIG. 5D). The enriched GO categories indicate that polyphenols modulate common regulatory processes, but the low similarity in their protein targets, illustrated by the low average JI, indicates that they target different processes within the same pathways.

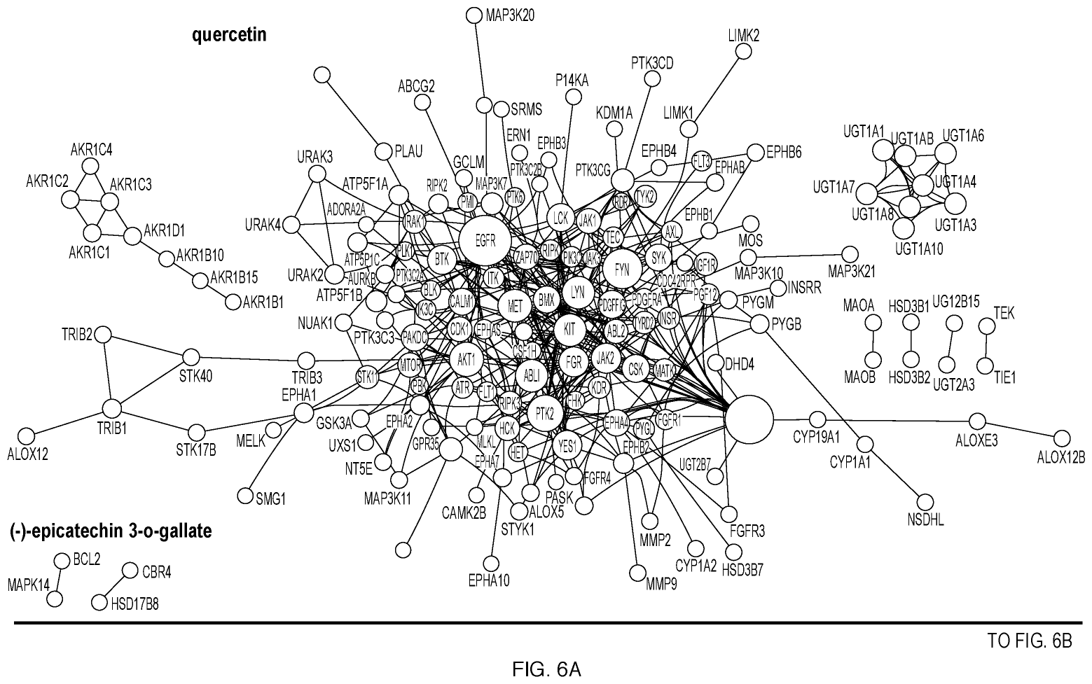

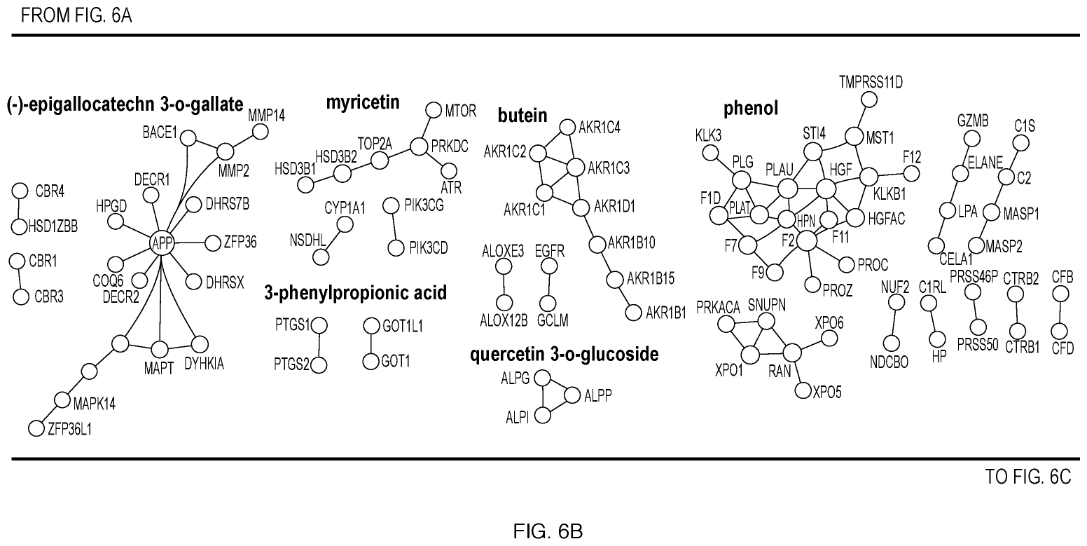

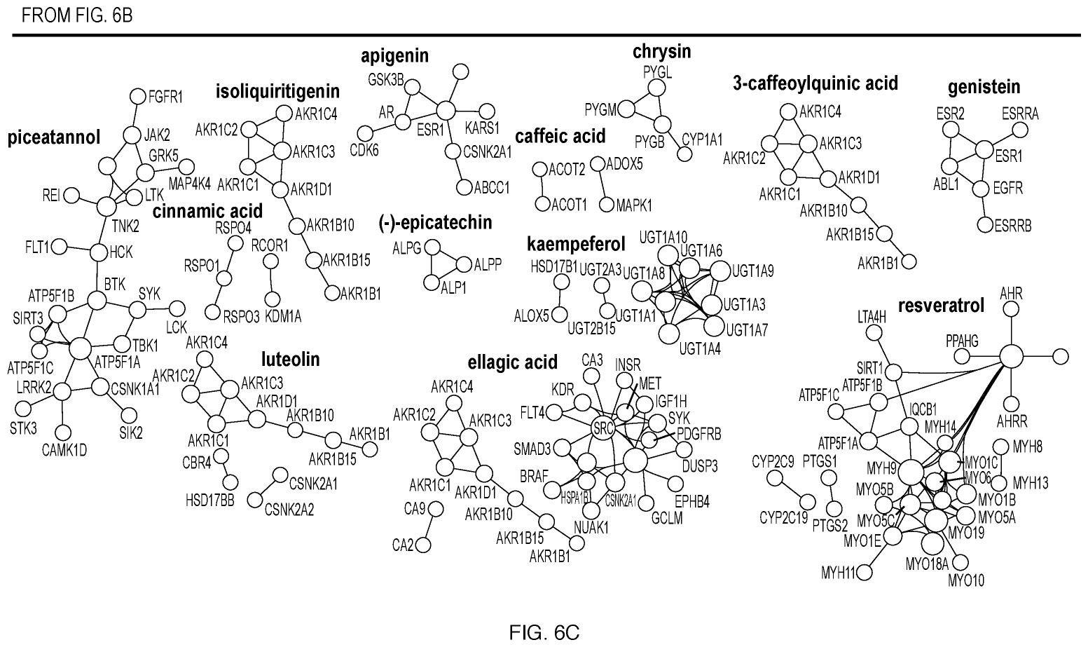

It was next asked whether the polyphenol targets cluster in specific regions of the human interactome. The focus was on polyphenols with more than two targets (n=46, FIG. 6) and measured the size and significance of the largest connected component (LCC) formed by the targets of each polyphenol. It was found that 25 of the 46 polyphenols have a larger LCC than expected by chance (Z-score>1.95) (FIG. 5E, FIG. 6). In agreement with experimental evidence documenting the effect of polyphenols on multiple pathways, it was found that ten polyphenols have their targets organized in multiple connected components of size >2. For example, the phenol targets, a compound with antiseptic and disinfectant properties, form three connected components with sizes 19, 6, 4 and 5 components of size 2 (FIG. 6).

Taken together, these results indicate that the targets of polyphenols modulate specific well localized neighborhoods of the interactome (FIG. 6).

### Example 3: Proximity Between Polyphenol Targets and Disease Proteins Reveals their Therapeutic Effects

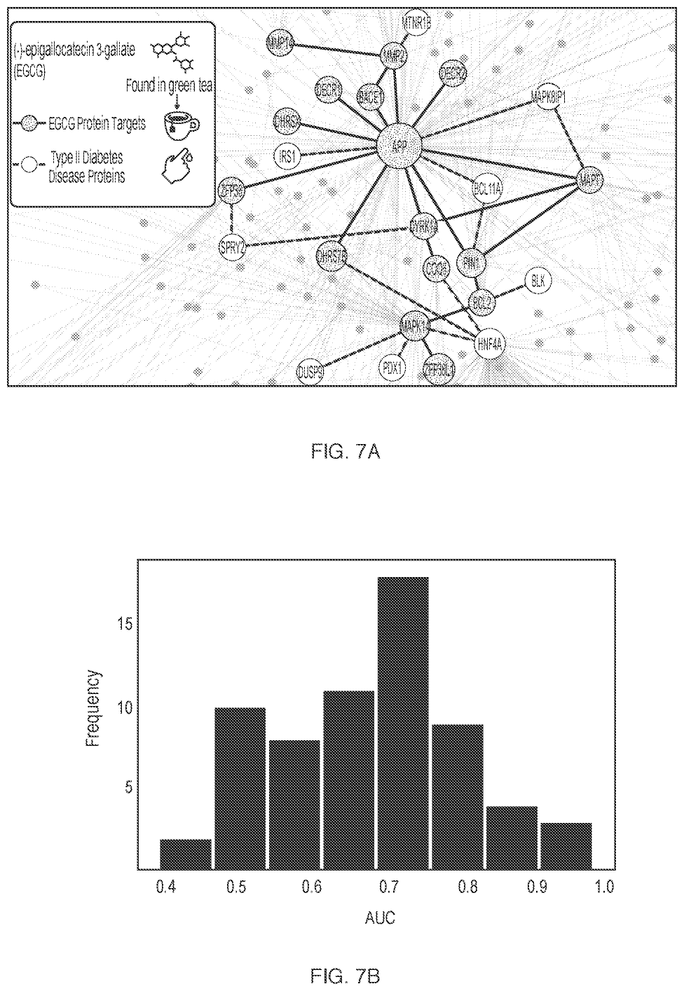

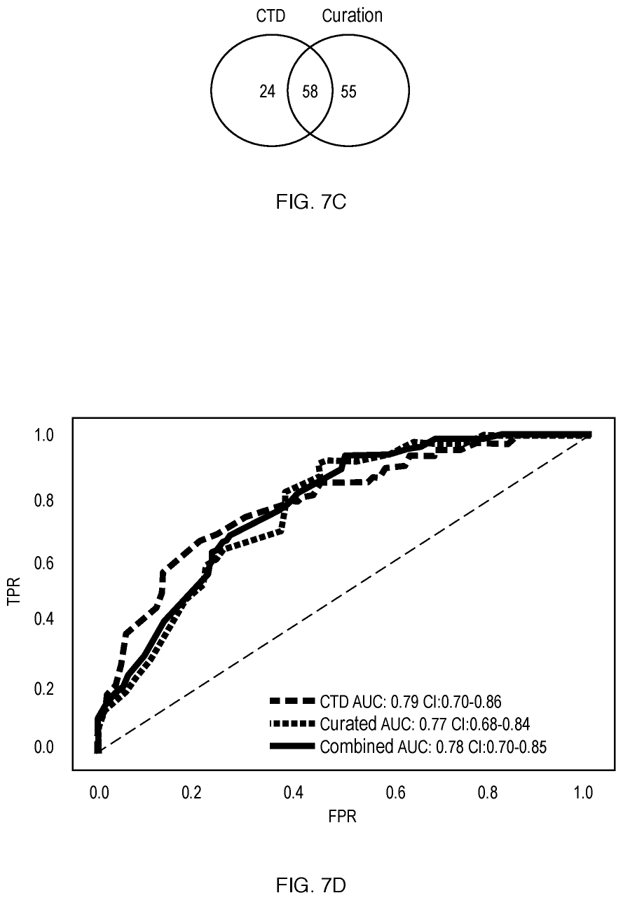

Polyphenols act like drugs: they bind to specific proteins, affecting their ability to perform their normal functions. The closer the targets of a polyphenol are to disease proteins, the more likely that the polyphenol will affect the disease phenotype, resulting in detectable therapeutic effects on the disease. The network proximity between polyphenol targets and proteins associated with 299 diseases was calculated using the closest measure, d_{c}, representing the average shortest path length between each polyphenol target and the nearest disease protein. Consider for example (−)-epigallocatechin 3-O-gallate (EGCG), a polyphenol abundant in green tea. Epidemiological studies have found a positive relationship between green tea consumption and reduced risk of type 2 diabetes mellitus (T2D), and physiological and biochemical studies have shown that EGCG presents glucose-lowering effects in both in vitro and in vivo models. Fifty-four experimentally validated EGCG protein targets were identified and mapped to the interactome, and it was found that the ECGC targets form an LCC of 17 proteins (Z=7.61) (FIG. 7A). The network-based distance between EGCG targets and 83 proteins associated with T2D was also computed, and it was found that the two sets are significantly proximal to each other. Indeed, several T2D proteins directly interact with the protein targets within the EGCG LCC (FIG. 7A). All 299 diseases were ranked based on the network proximity to the ECGC targets to determine if the 82 diseases in which ECGC has known therapeutic effects according to the CTD database could be recovered. The list recovered 15 previously known therapeutic associations among the top 20 ranked diseases (Table 1), confirming that network-proximity can discriminate between known and unknown disease associations for polyphenols, as previously confirmed in drugs. It was therefore demonstrated that the network proximity methods can be used to unveil novel therapeutic associations between food chemicals and diseases.

These methods were expanded to all polyphenol-disease pairs, with the goal of predicting diseases for which specific polyphenols might have therapeutic effects. For this, all possible 19,435 polyphenol-disease associations between 65 polyphenols and 299 diseases were grouped into known (1,525) and unknown (17,910) associations. The known polyphenol-disease set was retrieved from CTD, limiting to manually curated associations for which there is literature-based evidence. For each polyphenol, how well network proximity discriminates between the known and unknown sets was tested by evaluating the area under the Receiving Operating Characteristic (ROC) curve (AUC). For EGCG, network proximity offers a good discriminative power (AUC=0.78, CI: 0.70-0.86) between diseases with known and unknown therapeutic associations (Table 1). It was found that network proximity (d_{c}) offers predictive power with an AUC >0.7 for 31 polyphenols (FIG. 7B). In Table 2 the top 10 polyphenols for which the network medicine framework offered the best predictive power of therapeutic effects are summarized, the entries limited to prediction performance of AUC >0.6 and performance over top predictions with Precision >0.6.

Finally, multiple robustness checks were performed to rule out the role of potential biases in the input data. To test if the predictions are biased by the set of known associations retrieved from CTD, 100 papers were randomly selected from PubMed containing MeSH terms that tag EGCG to diseases. The evidence was manually curated for EGCG's therapeutic effects for the diseases discussed in the published papers, excluding reviews and non-English language publications. The dataset was processed to include implicit associations, resulting in a total of 113 diseases associated with EGCG, of which 58 overlap with the associations reported by CTD (FIG. 7C). It was observed that the predictive power of the network proximity was unchanged whether the annotations from CTD, the manually curated list, or the union of both (FIG. 7D) were considered. To test the role of potential biases in the interactome, the analysis was repeated using a subset of the interactome derived from an unbiased high-throughput screening and only high-quality polyphenol-protein interactions retrieved from ligand-protein 3D resolved structures. It was found that the predictive power was largely unchanged, indicating that the literature bias in the interactome does not affect the findings.

### Example 4: Network Proximity Predicts the Gene Expression Perturbation Induced by Polyphenols

To validate the predicted polyphenol-disease associations expression perturbation signatures were retrieved from the Connectivity Map database for the treatment of the breast cancer MCF7 cell line with 22 polyphenols. The database assigns each gene a z-score capturing the extent to which its expression is perturbed by a given polyphenol. The relationship between the extent in which polyphenols perturb the expression of disease genes, the network proximity between the polyphenol targets and disease proteins, and their known therapeutic effects was investigated (FIG. 8A). For example, different perturbation profiles for gene pools associated with different diseases were observed: for treatment with genistein (1 μM, 6 hours) 10 Skin Diseases (SD) genes with perturbation score >2 were observed, while only one highly perturbed Cerebrovascular Disorders (CD) was observed (FIG. 8B). Indeed, network proximity indicates that SD is closer to the genistein targets than CD, suggesting a relationship between network proximity, gene expression perturbation, and the therapeutic effects of the polyphenol (FIG. 8A). To test the validity of this hypothesis, an enrichment score was computed that measures the overrepresentation of disease genes among the most perturbed genes, finding 13 diseases that have their genes significantly enriched among the most deregulated genes by genistein, of which 4 have known therapeutic associations. It was found that these four diseases are significantly closer to the genistein targets than the nine diseases with non-therapeutic associations (FIG. 8C). A similar trend was observed for treatments with other polyphenols, whether the same (1 μM, FIG. 8C) or different (100 nM to 10 μM) concentrations were used. This result suggests that changes in gene expression caused by a polyphenol is indicative of its therapeutic effects, but only if the observed expression change is limited to proteins proximal to the polyphenol targets (FIG. 8A).

Network proximity can also be predictive of the overall gene expression perturbation caused by a polyphenol on the genes of a given disease. To test this, in each experimental combination defined by the polyphenol type and its concentration, the maximum perturbation score among genes for each disease was evaluated. The magnitude of the observed perturbation between diseases that were proximal (d_{c}<25th percentile, Z_{dc}<−0.5) or distal (d_{c}>75th percentile, Z_{dc}>−0.5) to the polyphenol targets were compared. FIGS. 9A and 9B show the results for the genistein treatment (1 μM, 6 hours), indicating that diseases proximal to the polyphenol targets show higher maximum perturbation values than distal diseases. The same trend was observed for other polyphenols (FIG. 9B), confirming that the impact of a polyphenol on cellular signaling pathways is localized in the network space, being greater in the vicinity of the polyphenol targets compared to neighborhoods remote from these targets.

Taken together, these results indicate that network proximity offers a mechanistic interpretation for the gene expression perturbations induced by polyphenols, being also predictive of whether these perturbations result in therapeutic effects.

### Example 5: Unveiling the Mechanisms Responsible for the Therapeutic Effects of Specific Polyphenols

How the network-based framework can facilitate the mechanistic interpretation of the therapeutic effects of selected polyphenols was demonstrated, with a focus on Vascular Diseases (VD). Out of 65 polyphenols evaluated in this study, 27 were found to have associations to VD, as their targets were hitting the VD network neighborhood (Table 3). The targets of 15 out of the 27 polyphenols with 10 or less targets were inspected, as experimentally validating the mechanism of action among the interactions of more than 10 targets would provide complexities beyond the scope of this study. The network analysis identified direct links between biological processes related to vascular health and the targets of three polyphenols, gallic acid, rosmarinic acid, and 1,4-naphthoquinone (FIG. 10).

Gallic Acid: Gallic acid has a single human protein target, SERPINE1, which is also a VD-associated protein, resulting in d_c=0 and Z_dc=−3.02. SERPINE1 is involved in the regulation of blood clot dissolution and regulation of cell adhesion and spreading by modulating the proteins PLAT and PLAU, respectively. An inspection of the LCC formed by VD proteins also revealed that SERPINE1 directly interacts with the VD proteins PLG, LRP1, and F2 (FIG. 10), proteins directly or indirectly related to blood clot formation and dissolution, suggesting that these pathways may be involved in potential gallic acid mechanism of action. Indeed, recent studies using in vivo models report that gallic acid has protective effects on vascular health.

1,4-Naphthoquinone: 1,4-naphthoquinone targets four proteins, MAP2K1, MAOA, CDC25B and IDO1, which are proximal to VD-associated proteins (d_c=1.25, [symbol] Z [symbol] _dc=−1.51) (FIG. 10). Indeed, the derivative compounds of 1,4-naphthoquinone have been explored as therapeutic agents for centuries. The polyphenol might influence biological processes related to vascular diseases through the action of its target MAP2K1, a gene involved in signaling pathways related to vascular smooth cell contraction and VEGF signaling, which also interacts with 5 VD associated proteins (FIG. 10). Mutations in MAP2K1 gene have been proposed as a cause of extracranial arteriovenous malformation as a result of endothelial cell dysfunction due to increased MEK1 activity. Additionally, one of 1,4-napthoquinone derivatives, shikonin, was shown to modulate inflammatory responses, protecting against brain ischemic damage.

Rosmarinic Acid: Rosmarinic acid (RA) can bind to three human proteins, FYN, MCL1, and AKR1B1, offering a statistically significant proximity to VD genes (d_c=1.00, [symbol] Z [symbol] _dc=−1.38). The analysis of the RA target FYN and three of its seven direct neighbors in the VD module (CD36, APP, and PRKCH) suggests the role of this polyphenol on platelet function—cells specialized in blood clot formation and involved in abnormal clotting that can lead to heart attacks and stroke. FYN also directly interacts with NFE2L2 (also known as NRF2), a transcription factor that regulates the expression of several genes with anti-oxidant properties43. Using RA perturbation profiles from the Connectivity Map database, it was observed that two cell lines (A549, MCF7) showed higher perturbation scores for genes that are directly regulated by NFE2L2 after treatment with RA. Indeed, recent reports show that mice lacking FYN have reduced platelet activit and that RA's protective effects on vascular calcification and on aortic endothelial function after diabetes-induced damage is mediated by anti-oxidant mechanisms. These observations suggest that RA activity might be mediated by FYN, ultimately regulating the processes of platelet activity and expression of anti-oxidant genes. The RA target MCL1 has also been proposed as an essential survival factor for endothelial cells in blood vessel production during angiogenesis, and it has been observed that RA has been found to restore cardiac function in rat models of ischemia/reperfusion injury.

In summary, by integrating literature evidence and by inspecting the polyphenol targets and their neighbors in the interactome, the molecular mechanisms underlying the protective effects of gallic acid, rosmarinic acid, and 1,4-naphthoquinone for VD were identified. The analysis suggests that gallic acid activity involves blood clot dissolution processes, rosmarinic acid acts on platelet activation and anti-oxidant pathways through FYN and its neighbors, and 1,4-naphthoquinone acts on signaling pathways of vascular cells through MAP2K1 activity.

### Example 6: Experimental Evidence Confirms that Rosmarinic Acid Modulates Platelet Function

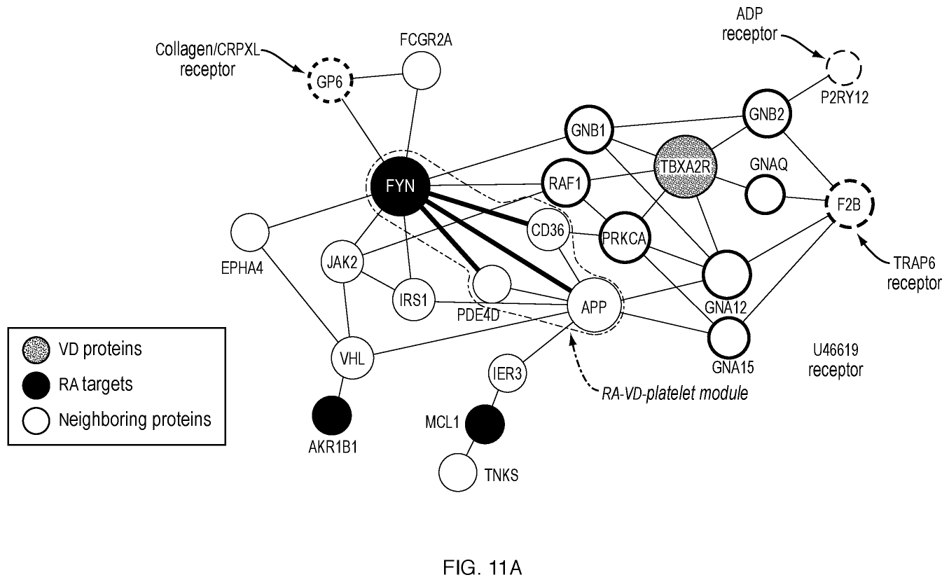

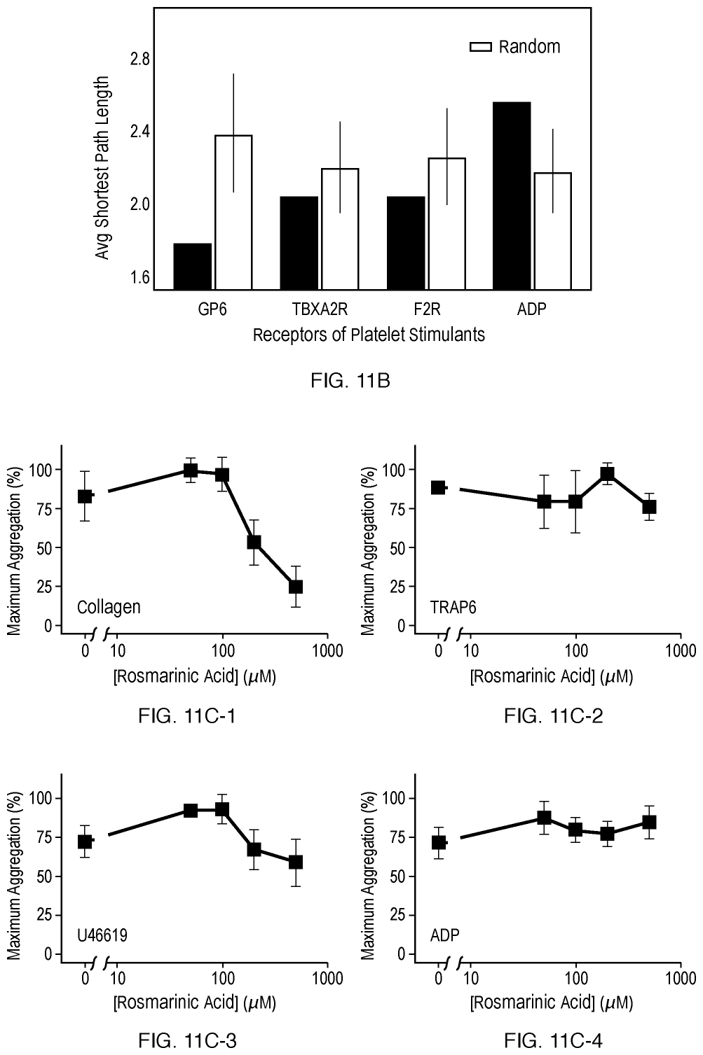

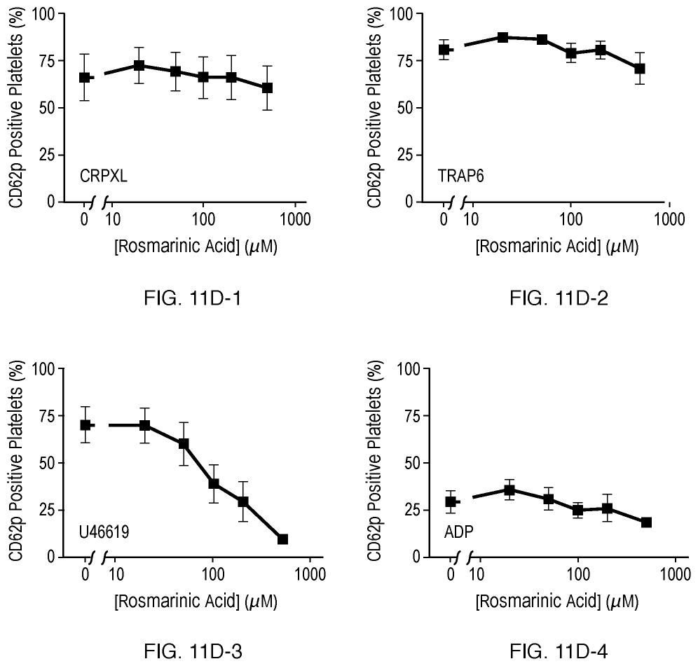

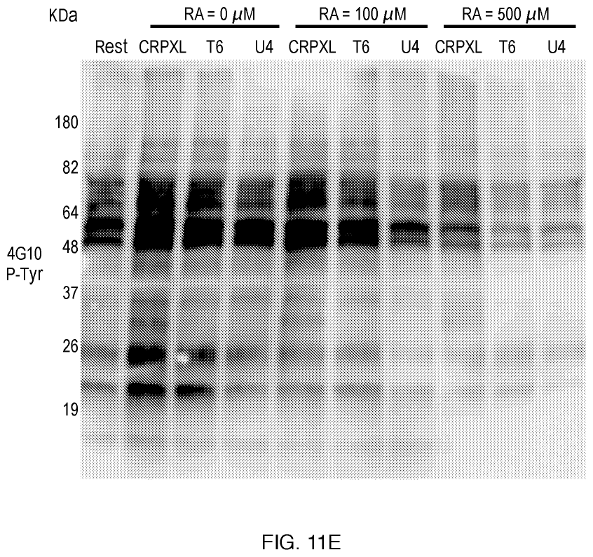

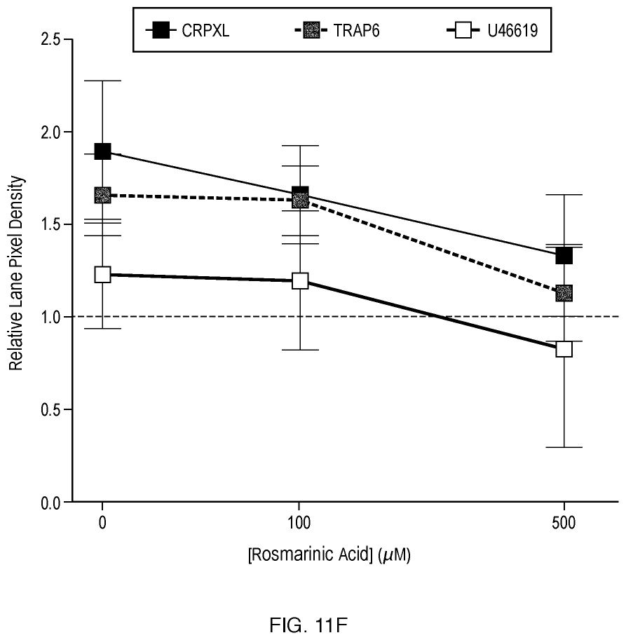

To validate the predictive power of the developed framework, direct experimental evidence of the predicted mechanistic role of Rosmarinic acid (RA) in VD was sought. The VD network neighborhood shows that RA targets are in close proximity to proteins related to platelet function, cells that control blood clot formation and whose inhibition is the mechanism underlying drugs prescribed to prevent heart attack and stroke. FIG. 11A shows the interactome region containing identified the RA-VD-platelet module: the connected component formed by the RA target FYN and the VD proteins associated to platelet function PDE4D, CD36, and APP; as well as its distance to the receptors of known platelet activators (FIG. 11A). Therefore, whether RA influenced platelet activation in vitro was evaluated. As platelets can be stimulated through different activation pathways, RA effects can, in principle, occur in any of them. To test these different possibilities, platelets were pretreated with RA and then activated with: 1) glycoprotein VI by collagen or collagen-related peptide (Collagen/CRPXL); 2) protease-activated receptors-1,4 by thrombin receptor activator peptide-6 (TRAP-6); 3) prostanoid thromboxane receptor by the thromboxane A2 analogue (U46619); and 4) P2Y1/12 receptor stimulation by adenosine diphosphate (ADP). When the network distance between each stimulant receptor and the RA-VD-platelet module (FIG. 11A) was compared, it was observed that the receptors for Collagen/CRPXL, TRAP-6, and U46619 are closer than the random expectation, while the receptor for ADP is more distant (FIG. 11B). It is expected that platelets would be most affected by RA when treated with stimulants whose receptors are most proximal to the RA-VD-platelet module, i.e. Collagen/CRPXL, TRAP-6, and U46619. As a control, no effect is expected for the distant receptor ADP. The experiments confirmed this prediction: RA inhibits collagen-mediated platelet aggregation (FIG. 11C) and impairs dense granule secretion induced by CRPXL, TRAP-6 and U46619. RA-treated platelets also displayed dampened alpha granule secretion (FIG. 11D) and integrin αIIbβ3 activation in response to U46619. As expected, RA did not affect platelet functions when a stimulant whose receptor is distant from the RA-VD-module was used. These findings suggest strong network effects is the way RA impairs several basic hallmarks of platelet activation, supporting that the proximity between RA targets and the functional neighborhood associated to platelet function (FIG. 11A) can explain RA impact on VD.

The molecular mechanisms involved in the functional impact of RA on platelets was clarified. The RA target FYN is a protein-tyrosine kinase and platelet activation is coordinated by several kinases that phosphorylate adaptors, enzymes, and cytoskeletal proteins downstream of platelet surface receptors. Given this connection, RA may inhibit platelets function by blocking agonist-induced protein tyrosine phosphorylation. It was observed that RA-treated platelets demonstrated a dose-dependent reduction in total tyrosine phosphorylation in response to CRPXL, TRAP-6 and U46619 (FIGS. 11E, 11F). This indicates that RA perturbs the phospho-signaling networks that regulate platelet response to extracellular stimuli.

Altogether, these findings support the prediction that RA, by targeting a network neighborhood related to platelet function, modulates platelet activation and function. It also supports the observation that its mechanism of action involves the protein-tyrosine kinase FYN (FIG. 11A) and the inhibition of tyrosine phosphorylation. Finally, while polyphenols are usually known for the health benefits caused by their antioxidant function, here another mechanism pathway through which they could benefit health is illustrated, in particular, by affecting platelet function.

### Example 7: Methods: Building the Interactome

The human interactome was assembled from 16 databases containing different types of protein-protein interactions (PPIs): 1) binary PPIs tested by high-throughput yeast-two-hybrid (Y2H) experiments; 2) kinase-substrate interactions from literature-derived low-throughput and high-throughput experiments from KinomeNetworkX, Human Protein Resource Database (HPRD), and PhosphositePlus; 3) carefully literature-curated PPIs identified by affinity purification followed by mass spectrometry (AP-MS), and from literature-derived low-throughput experiments from InWeb, BioGRID, PINA, HPRD, MINT, IntAct, and InnateDB; 4) high-quality PPIs from three-dimensional (3D) protein structures reported in Instruct, Interactome3D, and INSIDER; 5) signaling networks from literature-derived low-throughput experiments as annotated in SignaLink2.0; and 6) protein complex from BioPlex2.0. The genes were mapped to their Entrez ID based on the National Center for Biotechnology Information (NCBI) database as well as their official gene symbols. The resulting interactome includes 351,444 protein-protein interactions (PPIs) connecting 17,706 unique proteins. The largest connected component has 351,393 PPIs and 17,651 proteins.

### Example 8: Methods: Polyphenols, Polyphenol targets, and Disease Proteins

The 759 polyphenols were retrieved from the PhenolExplorer database. The database lists polyphenols with food composition data or profiled in biofluids after interventions with polyphenol-rich diets. For the analysis, only polyphenols that: 1) could be mapped in PubChem IDs, 2) were listed in the Comparative Toxicogenomics (CTD) database as having therapeutic effects on human diseases, and 3) had protein-binding information present in the STITCH database with experimental evidence were considered (FIG. 5A). After these steps, a final list of 65 polyphenols was considered, for which 598 protein targets were retrieved from STITCH. The 3,173 disease proteins considered corresponded to 299 diseases retrieved from Menche, J. et al. “Disease networks. Uncovering disease-disease relationships through the incomplete interactome.” Science 347, 1257601 (2015). Gene ontology enrichment analysis on protein targets was performed using the Bioconductor package clusterProfiler with a significance threshold of p<0.05 and Benjamini-Hochberg multiple testing correction with q<0.05.

### Example 9: Methods: Polyphenol Disease Associations

The polyphenol-disease associations were retrieved from the Comparative Toxicogenomics Database (CTD). Only manually curated associations labeled as therapeutic were considered. By considering the hierarchical structure of diseases along the MeSH tree, the study expanded explicit polyphenol-disease associations to include also implicit associations. This procedure was performed by propagating associations in the lower branches of the MeSH tree to consider also the diseases in the higher levels of the same tree branch. For example, a polyphenol associated with ‘heart diseases’ would also be associated to the more general category of ‘cardiovascular diseases’. By performing this expansion, a final list of 1,525 known associations between the 65 polyphenols and the 299 diseases considered in this study was obtained.

### Example 10: Methods: Network Proximity Between Polyphenol Targets and Disease Proteins

The proximity between a disease and a polyphenol was evaluated using a distance metric that takes into account the shortest path lengths between polyphenol targets and disease proteins. Given S, the set of disease proteins, T, the set of polyphenol targets, and d(s,t), the shortest path length between nodes s and tin the network, it is defined:

$${d}_{c} ( S , T ) = \frac{1}{‖ T ‖} {\sum }_{t ∈ T} {min}_{s ∈ S} d ( s , t ) \qquad [ 1 ]$$

To assess the significance of the distance between a disease and a polyphenol (S, T), a reference distance distribution was created corresponding to the expected distances between two randomly selected groups of proteins matching the size and degrees of the original disease proteins and polyphenol targets in the network. The reference distance distribution was generated by calculating the proximity between these two randomly selected groups, a procedure repeated 1,000 times. The mean μ_(d(S,T)) and s.d. σ_(d(S,T)) of the reference distribution were used to convert the absolute distance d_c to a relative distance Z_dc, defined as:

$${Z}_{d c} = \frac{d - {μ}_{{d}_{c} ( S , T )}}{{σ}_{{d}_{c} ( S , T )}} \qquad [ 2 ]$$

Due to the scale-free nature of the human interactome, there are few nodes with high degrees and to avoid repeatedly choosing the same (high degree) nodes, a degree-preserving random selection was performed.

### Example 11: Methods: Area Under ROC Curve Analysis

For each polyphenol, AUC was used to evaluate how well the network proximity distinguishes diseases with known therapeutic associations from all the others of the set of 299 diseases. The set of known associations (therapeutic) retrieved from CTD were used as positive instances, all unknown associations were defined as negative instances, and the area under the ROC curve was computed using the implementation in the Scikit-learn Python package. Furthermore, 95% confidence intervals were calculated using the bootstrap technique with 2,000 resamplings with sample sizes of 150 each. Considering that AUC provides an overall performance, a metric to evaluate the top-ranking predictions was used. For this analysis, the precision of the top 10 predictions was calculated, considering only the polyphenol-disease associations with relative distance Z_dc<−0.520.

### Example 12: Methods: Analysis of Network Proximity and Gene Expression Deregulation

Perturbation signatures were retrieved from the Connectivity Map database (https://clue.io/) for the MCF7 cell line after treatment with 22 polyphenols. These signatures reflect the perturbation of the gene expression profile caused by the treatment with that particular polyphenol relative to a reference population, which comprises all other treatments in the same experimental plate. For polyphenols having more than one experimental instance (time of exposure, cell line, dose), the one with highest distil_cc_q75 value (75th quantile of pairwise spearman correlations in landmark genes, https://clue.io/connectopedia/perturbagen\_types\_and\_controls) was selected. Gene Set Enrichment Analysis was performed to evaluate the enrichment of disease genes among the top deregulated genes in the perturbation profiles. This analysis offers an Enrichment Scores (ES) that have small values when genes are randomly distributed among the ordered list of expression values and high values when they are concentrated at the top or bottom of the list. The ES significance was calculated by creating 1,000 random selection of gene sets with the same size as the original set and calculating an empirical p-value by considering the proportion of random sets resulting in ES smaller than the original case. The p-values were adjusted for multiple testing using the Benjamini-Hochberg method. The network proximity d_c of disease proteins and polyphenol targets for diseases with significant ES were compared according to their therapeutic and non-therapeutic associations using the Student's t-test.

### Example 13: Methods: Platelet Isolation

Human blood collection was performed as previously described in accordance with the Declaration of Helsinki and ethics regulations with Institutional Review Board approval from Brigham and Women's Hospital (P001526). Healthy volunteers did not ingest known platelet inhibitors for at least 10 days prior. Citrated whole blood underwent centrifugation with a slow break (177 x g, 20 minutes) and the PRP fraction was acquired for subsequent experiments. For washed platelets, PRP was incubated with 1 μM prostaglandin E1 (Sigma, P5515) and immediately underwent centrifugation with a slow break (1000 x g, 5 minutes). Platelet-poor plasma was aspirated, and pellets resuspended in platelet resuspension buffer (PRB; 10 mM Hepes, 140 mM NaCl, 3 mM KCl, 0.5 mM MgCl2, 5 mM NaHCO3, 10 mM glucose, pH 7.4).

### Example 14: Methods: Platelet Aggregometry

Platelet aggregation was measured by turbidimetric aggregometry. Briefly, PRP was pretreated with RA for 1 hour before adding 250 μL to siliconized glass cuvettes containing magnetic stir bars. Samples were placed in Chrono-Log® Model 700 Aggregometers before the addition of various platelet agonists. Platelet aggregation was monitored for 6 minutes at 37° C. with a stir speed of 1000 rpm and the maximum extend of aggregation recorded using AGGRO/LINK®8 software. In some cases, dense granule release was simultaneously recorded by supplementing samples with Chrono-Lume® (Chrono-Log®, 395) according to the manufacturer's instructions.

### Example 15: Methods: Platelet Alpha Granule Secretion and Integrin αIIbβ3 Activation

Changes in platelet surface expression of P-selectin (CD62P) or binding of Alexa Fluor™ 488-conjugated fibrinogen were used to assess alpha granule secretion and integrin αIIbβ3 activation, respectively. First, PRP was pre-incubated with RA for 1 hour, followed by stimulation with various platelet agonists under static conditions at 37° C. for 20 minutes. Samples were then incubated with APC-conjugated anti-human CD62P antibodies (BioLegend®, 304910) and 100 μg/mL Alexa Fluor™ 488-Fibrinogen (Thermo Scientific™, F13191) for 20 minutes, before fixation in 2% [v/v] paraformaldehyde (Thermo Scientific™, AAJ19945K2). 50,000 platelets were processed per sample using a Cytek™ Aurora spectral flow cytometer. Percent-positive cells were determined by gating on fluorescence intensity compared to unstimulated samples.

### Example 16: Methods: Platelet Cytotoxicity

Cytotoxicity were tested by measuring lactate dehydrogenase (LDH) release by permeabilized platelets into the supernatant. Briefly, washed platelets were treated with various concentrations of RA for 1 hour, before isolating supernatants via centrifugation (15,000 x g, 5 min). A Pierce LDH Activity Kit (Thermo Scientific™, 88953) was then used to assess supernatant levels of LDH.

### Example 17: Methods: Platelet Phosphorylation

Washed platelets were pre-treated with RA for 1 hour, followed by agonist stimulation for 10 minutes. Platelets were lysed on ice with RIPA Lysis Buffer System® (Santa Cruz®, sc-24948) and sample supernatants clarified via centrifugation (14,000 rpm, 5 min, 4° C.). Supernatants were reduced with Laemmli Sample Buffer (Bio-Rad, 1610737) and proteins separated by molecular weight in PROTEAN TGX™ precast gels (Bio-Rad, 4561084). Proteins were transferred to PVDF membranes (Bio-Rad, 1620174) and probed with 4G10 (Milipore, 05-321), a primary antibody clone that recognizes phosphorylated tyrosine residues. Membranes were probed with horseradish peroxidase-conjugated secondary antibodies (Cell Signaling Technologies, 7074S) to catalyze an electrochemiluminescent reaction (Thermo Scientific™, PI32109). Membranes were visualized using a Bio-Rad ChemiDoc Imaging System and densitometric analysis of protein lanes conducted using ImageJ (NIH, Version 1.52a).

TABLE 1
Top 20 Predicted Therapeutic Associations Between
EGCG and Human Diseases

| | Distance | Significance |
|---|---|---|
| Disease | d_{c} | Z_{dc} |
| nervous system diseases | 1.13 | −1.72 |
| nutritional and metabolic diseases | 1.25 | −1.45 |
| metabolic diseases | 1.25 | −1.41 |
| cardiovascular diseases | 1.27 | −2.67 |
| immune system diseases | 1.29 | −1.31 |
| vascular diseases | 1.33 | −3.47 |
| digestive system diseases | 1.33 | −1.57 |
| neurodegenerative diseases | 1.37 | −1.71 |
| central nervous system diseases | 1.41 | −0.54 |
| autoimmune diseases | 1.41 | −1.30 |
| gastrointestinal diseases | 1.43 | −1.02 |
| brain diseases | 1.43 | −0.89 |
| intestinal diseases | 1.49 | −1.08 |
| inflammatory bowel diseases | 1.54 | −2.10 |
| bone diseases | 1.54 | −1.18 |
| gastroenteritis | 1.54 | −1.92 |
| demyelinating diseases | 1.54 | −1.78 |
| glucose metabolism disorders | 1.54 | −1.58 |
| heart diseases | 1.56 | −1.20 |
| diabetes mellitus | 1.56 | −1.66 |

Diseases were ordered according to the network distance (d_{c}) of their proteins to EGCG targets and diseases with relative distance Z_{dc} > −0.5 were removed.

TABLE 2
Top Ranked Polyphenols

| | | | | Concentration in | | N Mapped | LCC |
|---|---|---|---|---|---|---|---|
| Polyphenol | AUC | AUC CI* | Precision** | Blood*** | | Targets | Size |
| Coumarin | 0.93 | [0.86-0.98] | 0.6 | | | 7 | 1 |
| Piceatannol | 0.86 | [0.77-0.94] | 0.6 | | | 39 | 23 |
| Genistein | 0.82 | [0.75-0.89] | 0.7 | [0.006-0.525 | uM] | 18 | 6 |
| Ellagic acid | 0.79 | [0.63-0.92] | 0.6 | | | 42 | 19 |
| (−)-epigallocatechin 3-o-gallate | 0.78 | [0.70-0.86] | 0.8 | | | 51 | 17 |
| Isoliquiritigenin | 0.75 | [0.77-0.94] | 0.6 | | | 10 | 8 |
| Resveratrol | 0.75 | [0.66-0.82] | 1 | | | 63 | 25 |
| Pterostilbene | 0.73 | [0.61-0.84] | 0.6 | | | 5 | 2 |
| Quercetin | 0.73 | [0.64-0.81] | 1 | [0.022-0.080 | uM] | 216 | 140 |
| (−)-epicatechin | 0.65 | [0.49-0.80] | 0.8 | 0.625 | uM | 11 | 3 |

Table showing polyphenols with AUC > 0.6 and Precision > 0.6.
*Confidence intervals calculated with 2000 bootstraps with replacement and sample size of 50% of the diseases (150/299)
**Precision was calculated based on the top 10 polyphenols after their ranking based on the distance (d_{c}) of their targets to the disease proteins and considering only predictions with Z-score < −0.5.
***Concentrations of polyphenols in blood were retrieved from the Human Metabolome Database (HMDB)

TABLE 3
Polyphenols Proximal to Vascular Diseases

| | Number of | | |
|---|---|---|---|
| chemical | Protein Targets | d_{c} | Z_{dc} |
| gallic acid | 1 | 0.00 | −3.02 |
| prunetin | 1 | 0.00 | −2.82 |
| daidzin | 1 | 0.00 | −2.82 |
| punicalagin | 1 | 1.00 | −1.09 |
| kaempferol 3-o-galactoside | 1 | 1.00 | −1.75 |
| juglone | 2 | 1.00 | −1.92 |
| kaempferol 3-o-glucoside | 2 | 1.00 | −2.10 |
| 4-methylcatechol | 2 | 1.00 | −1.01 |
| rosmarinic acid | 3 | 1.00 | −1.38 |
| xanthotoxin | 3 | 1.33 | −2.05 |
| daidzein | 3 | 0.66 | −2.48 |
| umbelliferone | 3 | 1.33 | −1.50 |
| 1,4-naphtoquinone | 4 | 1.25 | −1.51 |
| 3-caffeoylquinic acid | 9 | 1.66 | −1.19 |
| isoliquiritigenin | 10 | 1.70 | −0.76 |
| chrysin | 12 | 1.50 | −0.64 |
| cinnamic acid | 15 | 1.46 | −1.37 |
| caffeic acid | 16 | 1.56 | −0.77 |
| genistein | 18 | 1.44 | −0.97 |
| 3-phenylpropionic acid | 18 | 1.72 | −0.53 |
| butein | 19 | 1.52 | −1.97 |
| myricetin | 34 | 1.47 | −0.60 |
| piceatannol | 39 | 1.05 | −2.64 |
| ellagic acid | 42 | 1.45 | −1.09 |
| (-)-epigallocatechin 3-o-gallate | 51 | 1.33 | −3.47 |
| phenol | 98 | 1.50 | −3.05 |
| quercetin | 216 | 1.37 | −2.18 |

The teachings of all references cited herein and in the attached paper are hereby incorporated in their entirety.

While example embodiments have been particularly shown and described, it will be understood by those skilled in the art that various changes in form and details may be made therein without departing from the scope of the embodiments encompassed by the appended claims.

1. A method of identifying a disease associated with a therapeutic chemical, comprising:
generating a candidate disease list based on proximities of proteins associated with a plurality of diseases and proteins associated with a therapeutic chemical in a protein-protein interaction network;
applying gene expression information associated with the therapeutic chemical to generate enrichment scores for diseases of the candidate disease list; and
identifying at least one disease associated with the therapeutic chemical based on the determined enrichment scores.

2. The method of claim 1, wherein generating the candidate disease list includes generating a proximity value for a disease and the therapeutic chemical.

3. The method of claim 2, wherein the proximity value is determined based on shortest path lengths between nodes representing proteins associated with the disease and nodes representing proteins associated with the therapeutic chemical.

4. The method of claim 3, wherein the proximity value is a distance metric d_{c}(S,T) determined according to:
$${d}_{c} ( S , T ) = \frac{1}{‖ T ‖} {\sum }_{t ∈ T} {min}_{s ∈ S} d ( s , t )$$
where S is a set of proteins associated with the disease, T is a set of proteins associated with the therapeutic chemical, s is a node representing a protein in set S, t is a node representing a protein in set T, and d(s, t) is a shortest path length between nodes s and t in the protein network.

5. A method of filtering data in a protein-protein interaction network, comprising:
mapping proteins associated with a plurality of diseases and proteins associated with a therapeutic chemical;
determining proximities of proteins associated with the plurality of diseases and proteins associated with the therapeutic chemical;
generating an enrichment score for each of the plurality of diseases based on gene expression information associated with the therapeutic chemical; and
generating a reduced dataset of proteins within the protein-protein interaction network, the reduced dataset of proteins being proteins associated with a subset of the plurality of diseases based on the determined proximities and the determined enrichment scores.

6. (canceled)

7. (canceled)

8. (canceled)

9. (canceled)

10. The method of claim 1, wherein generating an enrichment score includes measuring an extent of gene expression perturbation by the therapeutic chemical for a disease.

11. The method of claim 10, wherein measuring the extent of gene expression perturbation includes performing a Gene Set Enrichment Analysis.

12. (canceled)

13. The method of claim 1, further comprising ranking the diseases of the candidate disease list based on the determined proximity and the determined enrichment scores.

14. The method of claim 1, wherein the protein-protein interaction network is a human interactome.

15. The method of claim 1, wherein proteins associated with a therapeutic chemical are proteins to which the therapeutic chemical binds.

16. The method of claim 1, wherein the therapeutic chemical is a polyphenol and the proteins associated with the therapeutic chemical are binding targets of the polyphenol.

17. A method of treating a subject having a disease, the method comprising administering a therapeutic chemical, wherein the disease is a disease identified by the method of claim 1 as being associated with the therapeutic chemical.

18. (canceled)

19. A system for identifying a disease associated with a therapeutic chemical, comprising:
a processor configured to:
generate a candidate disease list based on proximities of proteins associated with a plurality of diseases and proteins associated with a therapeutic chemical in a protein-protein interaction network;
apply gene expression information associated with the therapeutic chemical to generate enrichment scores for diseases of the candidate disease list; and
identify at least one disease associated with the therapeutic chemical based on the determined enrichment scores.

20. The system of claim 19, wherein generating the candidate disease list includes generating a proximity value for a disease and the therapeutic chemical.

21. The system of claim 20, wherein the proximity value is determined based on shortest path lengths between nodes representing proteins associated with the disease and nodes representing proteins associated with the therapeutic chemical.

22. The system of claim 21, wherein the proximity value is a distance metric d_{c}(S,T) determined according to:
$${d}_{c} ( S , T ) = \frac{1}{‖ T ‖} {\sum }_{t ∈ T} {min}_{s ∈ S} d ( s , t )$$
where S is a set of proteins associated with the disease, T is a set of proteins associated with the therapeutic chemical, s is a node representing a protein in set S, t is a node representing a protein in set T, and d(s, t) is a shortest path length between nodes s and t in the protein network.

23. (canceled)

24. (canceled)

25. (canceled)

26. (canceled)

27. (canceled)

28. The system of claim 19, wherein generating an enrichment score includes measuring an extent of gene expression perturbation by the therapeutic chemical for a disease.

29. The system of claim 28, wherein measuring the extent of gene expression perturbation includes performing a Gene Set Enrichment Analysis.

30. (canceled)

31. The system of claim 19, wherein the processor is further configured to rank the diseases of the candidate disease list based on the determined proximity and the determined enrichment scores.

32. The system of claim 19, wherein the protein-protein interaction network is a human interactome.
